(19) United States
(12) Patent Application Publication
(10) Pub. No.: US 2020/0054771 A1
(43) Pub. Date: Feb. 20, 2020

(54) THERMOACOUSTIC IMAGING METHOD AND SYSTEM AND THERMOACOUSTIC IMAGING CONTRAST AGENT

(71) Applicant: ENDRA Life Sciences Inc., Ann Arbor, MI (US)

(72) Inventors: Michael M. Thornton, London (CA); Charlton Chen, Ann Arbor, MI (US); Paul A. Picot, London (CA); Marco A. Mena, Santa Clara, CA (US)

(73) Assignee: ENDRA Life Sciences Inc., Ann Arbor, MI (US)

(21) Appl. No.: 16/104,472

(22) Filed: Aug. 17, 2018

(51) Int. Cl. A61K 49/22

(52) U.S. Cl. CPC A61K 49/22

(57) ABSTRACT

A thermoacoustic imaging method comprises administering a thermoacoustic imaging contrast agent to a subject, the thermoacoustic imaging contrast agent having a viscosity higher than blood and substantially similar to a viscosity of a known computed tomography (CT) or magnetic resonance imaging (MRI) contrast agent; and imaging the subject with a thermoacoustic imaging system. The thermoacoustic imaging contrast agent may comprise an ionic solution and thickening agent mixture, with an optional heating agent. The ionic salt makes 0.5% to 5.0% of the ionic solution by weight and the remainder of the ionic solution is water. The thickening agent makes 3% to 50% of the mixture by weight.

### FIELD

The subject disclosure relates to a thermoacoustic imaging method and system and to a thermoacoustic imaging contrast agent.

### BACKGROUND

Blood vessel morphology and tissue perfusion can indicate states of health in organs and can be used for diagnosis of disease and monitoring of treatment. Measurement of blood flow in tissue is a key parameter in characterizing the type, state and/or health of tissue and can be used to diagnose several disorders or disease states including renal disease, cardiovascular disease, stroke, and cancer.

The differential filing of tissue of interest within a subject with an exogenous imaging agent, commonly referred to as a contrast agent, is often used in clinical practice to identify tissue abnormalities across multiple imaging modalities (nuclear imaging, magnetic resonance imaging (MRI), X-ray computed tomography (CT) imaging, ultrasound imaging, and positron emission tomography (PET) imaging). Typically, the contrast agent is administered to the subject prior to imaging by venous injection allowing the contrast agent to pass through blood vessels to an organ or tissue of interest within the subject. The contrast agent may remain in the blood pool or in some cases may migrate through blood vessel walls into interstitial spaces. Accepted tracer kinetics methods are used to estimate perfusion of the contrast agent thereby allowing blood flow characteristic data to be developed.

For example, in one common method of determining the perfusion of blood, an iodinated contrast agent is injected into the vasculature of a subject and thereafter, a sequence of X-ray computed tomography (CT) images of the subject are acquired allowing the progression of the iodinated contrast agent within the subject to be determined. With knowledge of the amount of injected iodinated contrast agent and a measure of the imaging waveform, blood flow, blood volume, and mean transit time within the subject can be estimated. The permeability-surface area product of the tissue of interest can also be estimated. Together, these measurements characterize the blood flow properties of tissue, which can be used to classify tissue, and can be used for diagnostic purposes.

Unfortunately, the above method has several drawbacks, including patient exposure to ionizing radiation, contrast agents that may not be physiologically tolerable, high operating costs of equipment, and large equipment requiring a specialized facility. Alternatively, a similar method of determining the perfusion of blood can be performed that employs magnetic resonance imaging to acquire images of the subject following injection of a suitable contrast agent into the vasculature of the subject in order to derive perfusion measurements. Unfortunately, this method suffers from many of the same drawbacks as X-ray computed tomography (CT) imaging.

Thermoacoustic imaging is an imaging modality that provides information relating to the thermoelastic properties of tissue. Thermoacoustic imaging uses short pulses of electromagnetic energy directed into a subject to heat absorbing features within the subject rapidly, which in turn induces acoustic pressure waves that are detected using acoustic receivers such as one or more thermoacoustic transducer arrays. The detected acoustic pressure waves are analyzed through signal processing, and processed for presentation and interpretation by an operator.

Although thermoacoustic imaging alleviates problems associated with X-ray CT imaging and MRI described above, typical contrast agents used for thermoacoustic imaging lack the rheological properties to maintain an extended residence time in the subject. Specifically, these contrast agents lack sufficient viscosity under the conditions found in the human body. As a result, the residence time, travel time, and time of efficacy of these contrast agents are unknown as these contrast agents are unfamiliar to medical professionals making blood flow, blood volume, and mean transit time estimation difficult.

As will be appreciated, improvements are desired. It is therefore an object at least to provide a novel thermoacoustic imaging method and system and a novel thermoacoustic imaging contrast agent.

### SUMMARY

It should be appreciated that this Summary is provided to introduce a selection of concepts in a simplified form that are further described below in the Detailed Description. This Summary is not intended to be used to limit the scope of the claimed subject matter.

Accordingly, in one aspect there is provided thermoacoustic imaging method comprising: administering a thermoacoustic imaging contrast agent to a subject, the thermoacoustic imaging contrast agent having a viscosity higher than blood and substantially similar to a known computed tomography or magnetic resonance imaging contrast agent; and imaging the subject with a thermoacoustic imaging system.

In one or more embodiments, the thermoacoustic imaging contrast agent comprises an ionic solution and thickening agent mixture, wherein an ionic salt comprises 0.5% to 5.0% of the ionic solution by weight and the remainder of the ionic solution is water, and wherein the thickening agent comprises 3% to 50% of the mixture by weight.

In one or more embodiments, the ionic salt is selected from the group consisting of sodium chloride, potassium chloride, magnesium chloride, calcium chloride, or some combination thereof.

In one or more embodiments, the thickening agent is a physiologically tolerable additive that increases the viscosity of the thermoacoustic imaging contrast agent. The thickening agent may be selected from the group consisting of dextran, albumin, hydroxyethyl starch, or some combination thereof.

In one or more embodiments, the thermoacoustic imaging contrast agent has a viscosity between 3 centipoise and 20 centipoise at 98 degrees Fahrenheit.

In one or more embodiments, the method further comprises heating the thermoacoustic imaging contrast agent to a desired temperature prior to administering the thermoacoustic imaging contrast agent to the subject. The desired temperature may for example be at or above the body temperature of the subject.

In one or more embodiments, the thermoacoustic imaging contrast agent further comprises a heating agent. The heating agent may be in an amount of 10 grams per liter of thermoacoustic imaging contrast agent or dilution thereof. The heating agent may be selected from the group consisting of gold nanoparticles, silver nanoparticles, platinum nanoparticles, iron-oxide nanoparticles, or some combination thereof.

In one or more embodiments, the method comprises, after administering the thermoacoustic imaging contrast agent to the subject, waling a predefined period of time before imaging the subject. The predefined period of time may for example be an estimated travel time of the thermoacoustic imaging contrast agent from its injection site in the subject to a region of interest within the subject to be imaged.

According to another aspect, there is provided a thermoacoustic imaging method comprising: administering to a subject a sufficient amount of a mixture that matches the viscosity of a known computed tomography or magnetic resonance imaging contrast agent, wherein the mixture comprises, an ionic solution, wherein an ionic salt comprises 0.5% to 5.0% of the ionic solution by weight and the remainder of the ionic solution is water, and a thickening agent, wherein the thickening agent comprises 3% to 50% of the mixture by weight; and imaging the subject with a thermoacoustic imaging system.

According to another aspect, there is provided a thermoacoustic imaging system comprising: an injector configured to deliver to a subject a thermoacoustic imaging contrast agent having viscosity characteristics substantially similar to those of a known computed tomography or magnetic resonance imaging contrast agent; an ultrasound receiving transducer or transducer array; a radiofrequency or microwave electromagnetic energy transmitter or transmitter array, wherein said electromagnetic energy transmitter or transmitter array is configured to administer modulated radiofrequency or microwave electromagnetic energy pulses to excite a thermoacoustic effect in soft tissue or vasculature of the subject; and hardware or a computer containing software to process thermoacoustic signals generated by the soft tissue or vasculature as a result of the thermoacoustic effect and generate a series of images based on the thermoacoustic contrast agent in the soft tissue or vasculature over time.

### BRIEF DESCRIPTION OF THE DRAWINGS

Embodiments will now be described more fully with reference to the accompanying drawings in which:

FIG. 1 depicts a thermoacoustic imaging system;

FIG. 2 depicts a conformal, flexible electromagnetic source applicator forming part of the thermoacoustic imaging system of FIG. 1;

FIG. 3 is a graph of the intrinsic absorption properties of different types of tissue as a function of electromagnetic frequency;

FIG. 4 is a graph of the absorption properties of different tissues and contrast agents as a function of electromagnetic frequency;

FIG. 5 shows thermoacoustic data following pulsed radiofrequency irradiation of: a 2-mm tube containing physiological 0.9% saline surrounded by de-ionized water (upper left panel), a 2-mm tube containing 2% saline surrounded by physiological 0.9% saline (upper right panel); a 2-mm tube and a 3-mm containing de-ionized water surrounded by physiological 0.9% saline (lower left panel); and 2-mm tube containing light mineral oil surrounded by water (lower right panel);

FIG. 6 is an image of thermoacoustic data following pulsed radiofrequency irradiation of four 0.3-mm tubes containing 5× physiological saline (5% NaCl) surrounded by physiological 0.9% saline;

FIG. 7 shows the relationship between changes in whole blood viscasity and blood flow in a human cal muscle during and after infusions of low molecular weight dextran and Hartmann's solution;

FIG. 8 shows the viscosity of a 3.6% saline mixture as a function of albumin polypeptide in the mixture;

FIG. 9 shows the viscosity of a 3.0 molal saline mixture as a function of albumin polypeptide in the mixture;

FIG. 10 shows the viscosity of a water mixture as a function 2-hydroxyethyl starch in the mixture;

FIG. 11 shows the viscosity of hot-mixed and cold-mixed 2-hydroxyethyl starch mixtures;

FIG. 12 shows real permittivity as a function of weight percent albumin polypeptide;

FIG. 13 shows imaginary permittivity as a function of weight percent albumin polypeptide;

FIG. 14 shows thermoacoustic response as a function of saline solution conductivity with nanoparticle sized heating agents; and

FIG. 15 shows a method of performing thermoacoustic imaging on a subject with a viscous contrast agent mixture.

### DETAILED DESCRIPTION DO THE EMBODIMENTS

The foregoing summary, as well as the following detailed description of certain examples will be better understood when read in conjunction with the appended drawings. As used herein, an element or feature introduced in the singular and preceded by the word “a” or “an” should be understood as not necessarily excluding the plural of the elements or features. Further, references to “one example” or “one embodiment” are not intended to be interpreted as excluding the existence of additional examples or embodiments that also incorporate the described elements or features. Moreover, unless explicitly stated to the contrary, examples or embodiments “comprising” or “having” or “including” an element or feature or a plurality of elements or features having a particular property may include additional elements or features not having that property. Also, it will be appreciated that the terms “comprises”, “has”, “includes” means “including but not limited to” and the terms “comprising”, “having” and “including” have equivalent meanings.

As used herein, the term “and/or” can include any and all combinations of one or more of the associated listed elements or features.

It will be understood that when an element or feature is referred to as being “on”, “attached” to, “connected” to, “coupled” with, “contacting”, etc. another element or feature, that element or feature can be directly on, attached to, connected to, coupled with or contacting the other element or feature or intervening elements may also be present. In contrast, when an element or feature is referred to as being, for example, “directly on”, “directly attached” to, “directly connected” to, “directly coupled” with or “directly contacting” another element of feature, there are no intervening elements or features present.

It will be understood that spatially relative terms, such as “under”, “below”, “lower”, “over”, “above”, “upper”, “front”, “back” and the like, may be used herein for ease of description to describe the relationship of an element or feature to another element or feature as illustrated in the figures. The spatially relative terms can however, encompass different orientations in use or operation in addition to the orientation depicted in the figures.

Thermoacoustic imaging, a general term encompassing photoacoustic, optoacoustic, and photo-thermoacoustic imaging, is a field of technology used in characterizing and imaging heat absorbing features (tissue and/or vasculature) within a subject based on their electromagnetic absorption and thermal properties. To date, most other imaging modalities measure the same energy that is used as the input. For example, optical systems input and receive light, ultrasound systems input and receive ultrasound, X-ray computed tomography systems input and receive X-rays, and magnetic resonance systems input and receive radiofrequency energy. Thermoacoustic imaging however, is a hybrid modality which inputs electromagnetic energy but receives acoustic energy.

During thermoacoustic imaging, pulses of electromagnetic (EM) energy are transmitted and directed into a subject and absorbed by the tissue and/or vasculature within the subject to be imaged. Typically, near-infrared, microwave, or radiofrequency electromagnetic waves are used. The absorbed electromagnetic energy causes immediate heating, thermal expansion, and generation of acoustic pressure waves with temporal characteristics defined by the incident pulses of electromagnetic energy. The acoustic pressure waves are detected using one or more thermoacoustic transducer arrays and are analyzed through signal processing, and processed for presentation and interpretation by an operator.

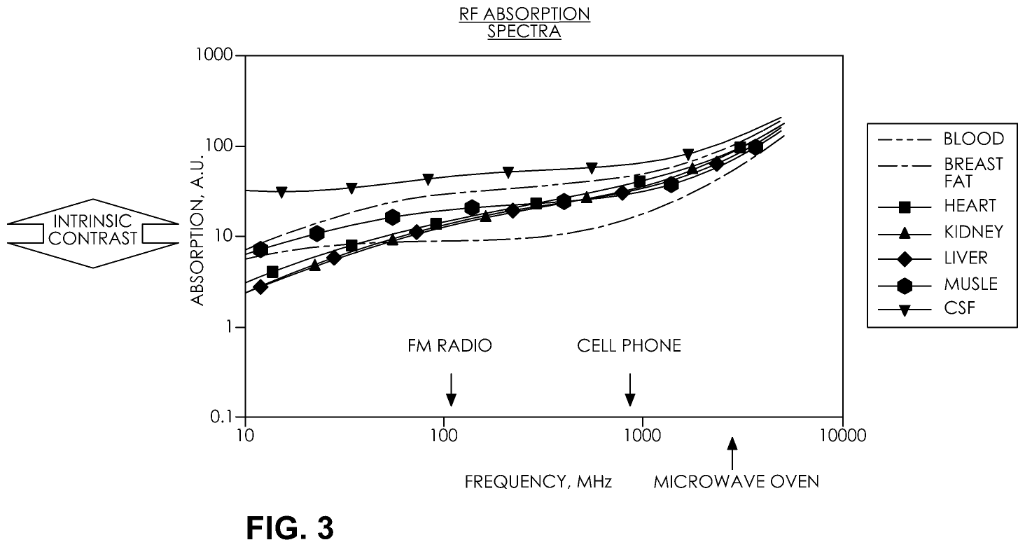

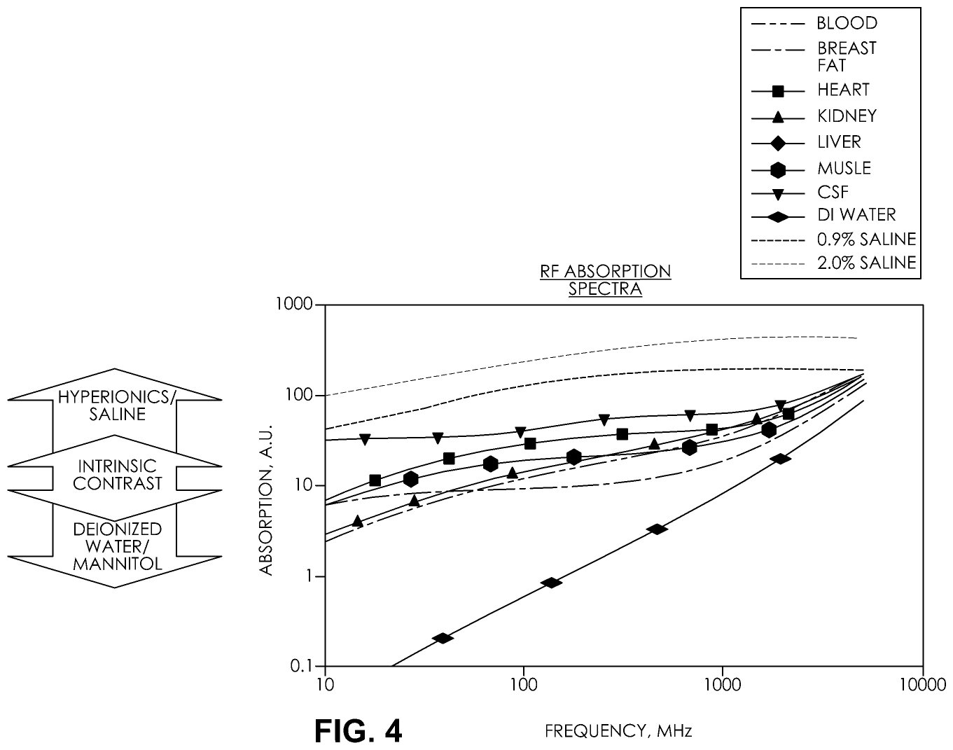

Thermoacoustic imaging provides a spatial map of the relative energy absorption by the tissue and/or vasculature. In the radio and microwave frequencies, endogenous energy absorption by tissue is dominated by ion concentration and dielectric absorption. The soft tissue contrast may be increased by the introduction of an exogenous vascular contrast agent that either increases or decreases the absorption rate of the irradiating electromagnetic energy by the tissue. The contrast agents are physiologically tolerable, that is, the contrast agents do not cause immediate or lasting deleterious effects to living organisms and are generally regarded as safe (GRAS) as defined by the U.S. Food and Drug Administration (FDA). FIG. 3 depicts the absorption properties of several different tissues. Contrast agents differ in the absorption properties depending on the application and the wavelength of the electromagnetic energy (FIG. 4).

For example, a contrast agent with an ion concentration that is hyperionic compared to blood will increase the absorption rate of radiofrequency (RF) radiation by tissue containing the contrast agent, while a contrast agent with hypotonic ion concentration compared to blood will decrease the absorption rate of RF radiation by tissue containing the contrast agent. Alternatively, the introduction of a contrast agent that has lower dielectric loss than water will decrease the absorption rate of microwave energy by tissue containing the contrast agent. Similarly, a contrast agent with higher dielectric loss than water will increase the absorption rate of microwave energy by tissue containing the contrast agent.

It has been found that suitable contrast agents in connection with thermoacoustic imaging yielding the desired increase in soft tissue contrast are those having an increased dielectric constant compared to soft tissue or blood (e.g., a dielectric constant that is at least 1.5-fold, 2.0-fold, 3.0-fold, 4.0-fold, 5.0-fold, 6.0-fold, 7.0-fold, 8.0-fold, 9.0-fold, 10-fold, 15-fold, 20-fold, 25-fold, 30-fold, 35-fold, 40-fold, 45-fold, 50-fold, 60-fold, 70-fold, 80-fold, 90-fold, or 100-fold greater than the dielectric constant of soft tissue or blood), or those having an increased ionic conductivity compared to soft tissue or blood (e.g., ionic conductivity that is at least 1.5-fold, 2.0-fold, 3.0-fold, 4.0-fold, 5.0-fold, 6.0-fold, 7.0-fold, 8.0-fold, 9.0-fold, 10-fold, 15-fold, 20-fold, 25-fold, 30-fold, 35-fold, 40-fold, 45-fold, 50-fold, 60-fold, 70-fold, 80-fold, 90-fold, or 10-fold greater than the ionic conductivity of soft tissue or blood).

Other suitable contrast agents in connection with thermoacoustic imaging that yield the desired increase in soft tissue contrast are those having a decreased dielectric absorption compared to soft tissue or blood (e.g., a dielectric constant that is at least 1.5-fold, 2.0-fold, 3.0-fold, 4.0-fold, 5.0-fold, 6.0-fold, 7.0-fold, 8.0-fold, 9.0-fold, 10-fold, 15-fold, 20-fold, 25-fold, 30-fold, 35-fold, 40-fold, 45-fold, 50-fold, 60-fold, 70-fold, 80-fold, 90-fold, or 100-fold less than the dielectric absorption of soft tissue or blood) or those having a decreased ionic conductivity compared to soft tissue or blood (e.g., an ionic conductivity that is at least 1.5-fold, 2.0-fold, 3.0-fold, 4.0-fold, 5.0-fold, 6.0-fold, 7.0-fold, 8.0-fold, 9.0-fold, 10-fold, 15-fold, 20-fold, 25-fold, 30-fold, 35-fold, 40-fold, 45-fold, 50-fold, 60-fold, 70-fold, 80-fold, 90-fold, or 100-fold less than the ionic conductivity of soft tissue or blood). Those of skill in the art will appreciate that the above discussion of contrast agents for thermoacoustic imaging is exemplary and that other suitable contrast agents that rely on other mechanisms to modify (increase or decrease) electromagnetic energy absorption in tissue or the thermoacoustic efficiency of tissue, such as changing the temperature or ion concentration, may be used during thermoacoustic imaging.

US. Patent Application Publication No. US20120197117A1 to Picot at al. (“Picot”) discloses thermoacoustic imaging methods and systems and thermoacoustic imaging contrast agents for use during thermoacoustic imaging to increase soft tissue contrast. Picot is incorporated by reference into this disclosure in its entirety and portions of Picot have been restated herein. Although Picot discloses a variety of thermoacoustic imaging contrast agents to increase soft tissue contrast, the disclosed thermoacoustic imaging contrast agents lack the rheological properties to maintain an extended residence time in a subject. More particularly, the thermoacoustic imaging contrast agents lack sufficient viscosity under the conditions found in the human body.

The subject disclosure differentiates itself from Picot in a novel and non-obvious way by utilizing a thermoacoustic imaging contrast agent with a viscosity higher than blood. Further, the viscosity of the thermoacoustic imaging contrast agent is set to match substantially the viscosity of a known contrast agent utilized for X-ray computed tomography (CT) or magnetic resonance imaging (MRI). Since medical professionals are familiar with utilizing CT and MRI contrast agents, they are readily able to administer and utilize a thermoacoustic imaging contrast agent that substantially matches the rheological properties (viscosity) of a known CT or MRI contrast agent. Hence, functions such as residence time, travel time (e.g. bolus arrival time), and time of efficacy of the thermoacoustic imaging contrast agent can be accurately estimated by medical professionals based upon their experience gained from using known CT or MRI contrast agents. Examples of acceptable CT and MRI contrast agents are those contrast agents that have been approved by the U.S. FDA for the purpose of medical imaging. These CT and MRI contrast agents typically have a viscosity anywhere from 1 centipoise to 20 centipoise, depending upon the contrast agent. A partial and non-limiting list includes: AK-FLUOR 10% and 25%; CHOLOGRAFIN MEGLUMINE; CONRAY 30; CONRAY 43; CONRAY; CYSTO-CONRAY II; CYSTO-CONRAY; CYSTOGRAFIN DILUTE; CYSTOGRAFIN; DATSCAN; DEFINITY; GASTROGRAFIN; GASTROMARK; MAGNEVIST; MPI INDIUM DTPA IN 111; OMNISCAN; OPTIMARK; OPTIMARK IN PLASTIC CONTAINER; OPTISON; RENOGRAFIN-76; TECHNESCAN MAG3; TECHNESCAN PYP KIT; TECHNETIUM (99m Tc); FANOLESOMAB; NEUTROSPEC; TECHNETIUM TC 99M ALBUMIN AGGREGATED KIT; TECHNETIUM TC 99M SESTAMIBI (Generic Drug); TECHNETIUM TC 99M SESTAMBI (Generic Drug); TECHNETIUM TC 99M SESTAMIBI (Generic Drug); XENON XE 133; ABLAVAR; ADREVIEW; AMMONIA N 13; AMYVID; AN-DTPA; AN-SULFUR COLLOID; CARDIOGEN-82; CARDIOLITE; CEA-SCAN; CERETEC; CHOLETEC; CHOLINE C-11; CHROMITOPE SODIUM; CIS-MDP CIS-PYRO; DRAXIMAGE MDP-25; DTPA; EOVIST; ETHIODOL; FERIDEX I.V.; FLUDEOXYGLUCOSE F 18; FLUORESCITE; GADAVIST; GALLIUM CITRATE GA 67; GALLIUM CITRATE GA 67; GLOFIL-125; HEPATOLITE; HEXABRIX; HICON; IC-GREEN; INDICLOR; INDIUM IN 111 CHLORIDE; INDIUM IN-111 OXYQUINOLINE; INDOCYANINE GREEN; IOPAMIDOL-200 (Generic Drug); IOPAMIDOL-250 (Generic Drug); IOPAMIDOL-300 (Generic Drug); IOPAMIDOL-370 (Generic Drug); IOPAMDOL-370 IN PLASTIC CONTAINER (Generic Drug); ISOVUE-200; ISOVUE-250; ISOVUE-300; ISOVUE-370; ISOVUE-M 200; ISOVUE-M 300; JEANATOPE; MD-76R; MD-GASTROVIEW; MD-GASTROVIEW (Generic Drug); MDP-BRACCO; MEGATOPE; MPI DMSA KIDNEY REAGENT; MULTIHANCE; MULTIHANCE MULTIPACK; MYOVIEW; NEUROLITE; OCTREOSCAN; OMNIPAQUE 140; OMNIPAQUE 180; OMNIPAQUE 240; OMNIPAQUE 300; OMNIPAQUE 350; OPTIRAY 240; OPTIRAY 300; OPTIRAY 320; OPTIRAY 350; OXILAN-300; OXILAN-350; PROHANCE; PROHANCE; ULTIPACK; PROSTASCINT; PULMOLITE; SCANLUX-300 (Generic Drug); SCANLUX-370 (Generic Drug); SINOGRAFIN; SODIUM IODIDE I 131; TECHNELITE; THALLOUS CHLORIDE TL 201; THALLOUS CHLORIDE TL 202; THALLOUS CHLORIDE TL 203; ULTRATAG; ULTRA-TECHNEKOW FM; ULTRAVIST (PHARMACY BULK); ULTRAVIST 150; ULTRAVIST 240; ULTRAVIST 300 IN PLASTIC CONTAINER; ULTRAVIST 300; ULTRAVIST 370; VERLUMA; VISIPAQUE 270; and VISIPAQUE 320.

The subject disclosure describes thermoacoustic imaging methods and systems and thermoacoustic imaging contrast agents. Broadly stated, the subject thermoacoustic imaging methods described herein comprise administering a thermoacoustic imaging contrast agent to a subject, the thermoacoustic imaging contrast agent having a viscosity higher than blood and substantially similar to a known computed tomography or magnetic resonance imaging contrast agent; and imaging the subject with a thermoacoustic imaging system.

In one embodiment as shown in FIG. 15, the thermoacoustic imaging method comprises administering to a subject a sufficient amount of a mixture that matches the viscosity of a known computed tomography or magnetic resonance imaging contrast agent, wherein the mixture comprises: an ionic solution, wherein an ionic salt comprises 0.5% to 5.0% of the ionic solution by weight and the remainder of the ionic solution is water; and a thickening agent, wherein the thickening agent comprises 3% to 50% of the mixture by weight (step 15604); and imaging the subject with a thermoacoustic imaging system (step 1506).

The thickening agent is a physiologically tolerable additive that increases the viscosity of the mixture. For example, the thickening agent may be selected from the group consisting of dextran, album, hydroxyethyl starch, or some combination thereof.

The ionic salt may for example be selected from the group consisting of sodium chloride, potassium chloride, magnesium chloride, or some combination thereof.

During formulation of the thermoacoustic imaging contrast agent, the amount of thickening agent used in the mixture is selected until the viscosity of the mixture substantially matches the viscosity of a CT or MRI contrast agent known to the medical professional conducting the thermoacoustic imaging procedure. Typically, the viscosity of the mixture is in the range of 3 centipoise to 20 centipoise at 98 degrees Fahrenheit. Depending on the amount of thickening agent used to establish the desired viscosity of the mixture, the amount of ionic salt used in the ionic solution is adjusted so that the dielectric constant of the mixture compared to the dielectric constant of the tissue and/or vasculature to be imaged is at the desired ratio so that the desired increase in soft tissue contrast is achieved.

A higher viscosity contrast agent has two major benefits. First, the travel of the contrast agent through a desired volume or region of interest within the subject is slower due to the higher viscosity. This results in a longer residence time of the contrast agent in the desired volume or region of interest and greater opportunity to image the desired volume or region of interest of the subject.

A second benefit is that the bolus, or initial ball of contrast agent injected into the subject, does not disperse as quickly. This also results in the contrast agent remaining in the desired volume or region of interest for a longer period of time. Hence, there is a greater opportunity to image the desired volume or region of interest of the subject.

The relationship between viscosity and blood flow was explored by Dormand (British Medical Journal, 18 Dec. 1971, 4, pp. 716-719.). Dormand slated that there are three major determinants in peripheral blood flow: perfusion pressure, the morphology of the vessels, and viscosity of the blood. Dormand determined blood flow differences after changing viscosity with infused solutions of dextran and Hartmann's solution. The comparison between dextran and Hartmann's solution showed that the effect of haemodilution is negligible. Hence, viscosity is a primary factor in the rate of peripheral blood flow.

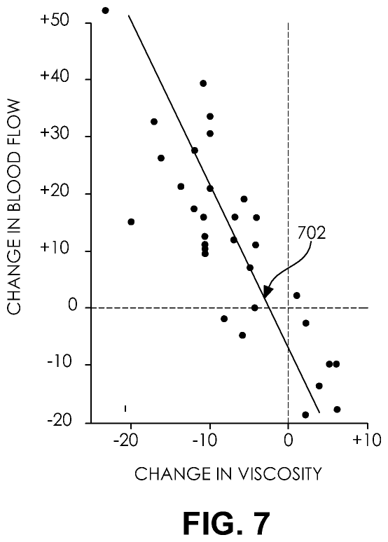

FIG. 7 shows the relationship between changes in whole blood viscosity and blood flow in a human cat muscle during and after infusions of low molecular weight dextran and Hartmann's solution. The relationship shows that a higher viscosity results in a lower blood flow rate, as shown with a line 702 that interpolates the data points.

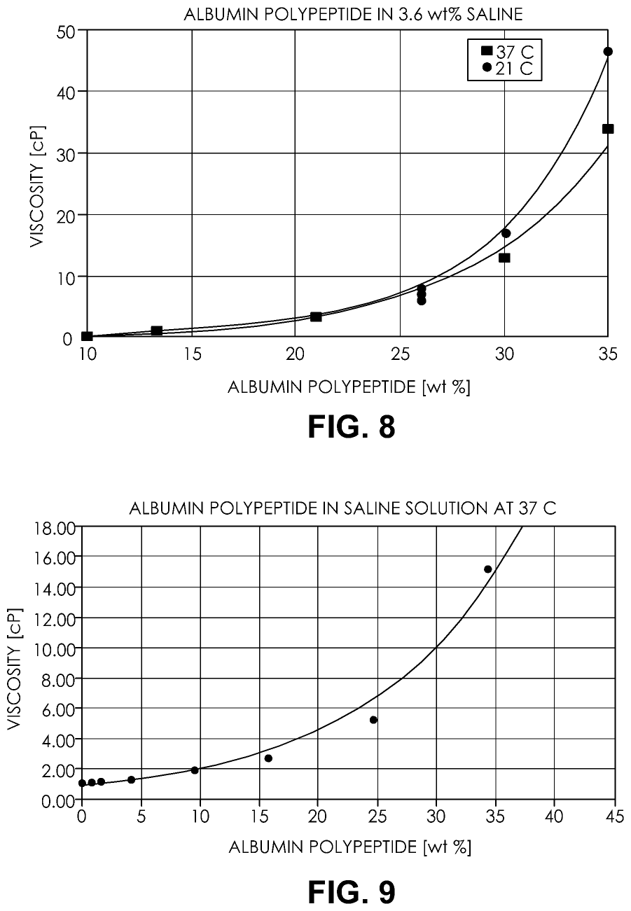

FIG. 8 shows the viscosity of a 3.6% saline mixture as a function of albumin polypeptide in the mixture. Interpolated curve 802 shows the data at 37 degrees Celsius (body temperature). Interpolated curve 804 shows the data at 21 degrees Celsius (room temperature).

FIG. 9 shows the viscosity of a 3.0 molal saline mixture as a function of albumin polypeptide in the mixture. Interpolated curve 902 shows that viscosity increases as more thickener, albumin polypeptide, is added.

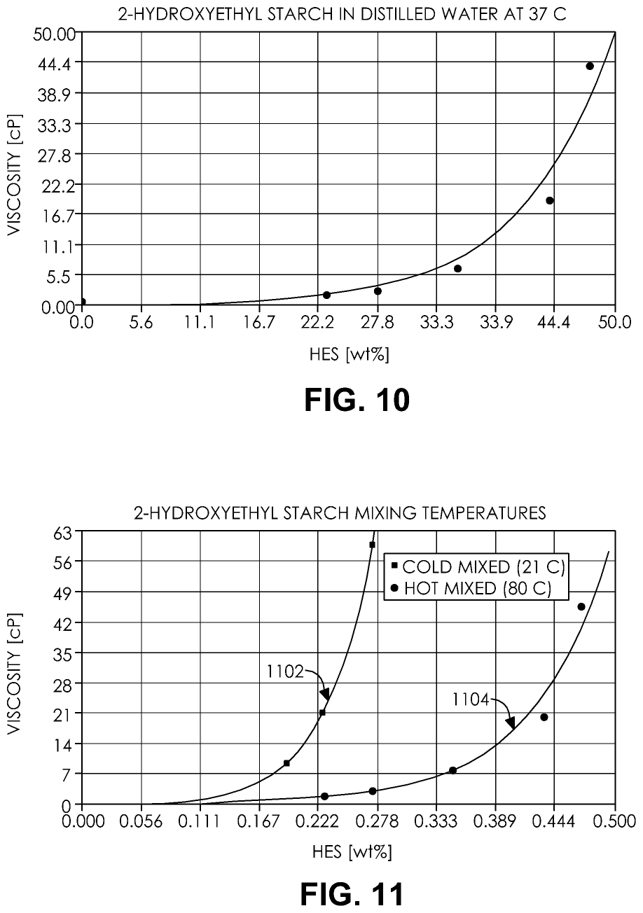

FIG. 10 shows the viscosity of a water mixture as a function 2-hydroxyethyl starch in the mixture. Interpolated curve 1002 shows that viscosity increases as more thickener, 2-hydroxyethyl starch, is added.

FIG. 11 shows the viscosity of hot-mixed and cold-mixed 2-hydroxyethyl starch mixtures. Interpolated curve 1102 shows that viscosity increases as more hot-mixed thickener, 2-hydroxyethyl starch, is added. Interpolated curve 1104 shows that viscosity increases as more hot-mixed thickener, 2-hydroxyethyl starch, is added.

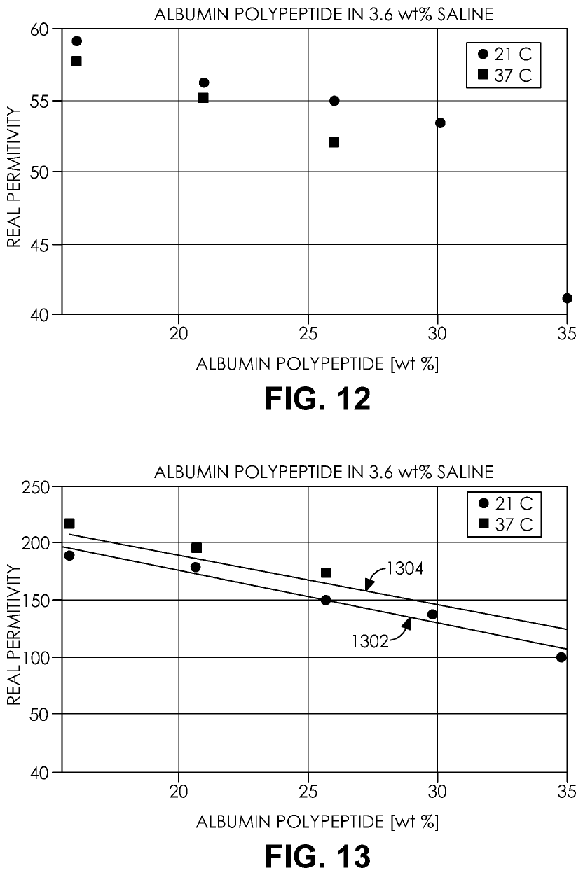

FIG. 12 shows real permittivity as a function of weight percent albumin polypeptide.

FIG. 13 shows imaginary permittivity as a function of weight percent albumin polypeptide. Regression line 1302 shows that imaginary permittivity decreases as albumin polypeptide increases at 21 degrees Celsius. Regression line 1304 shows that imaginary permittivity decreases as albumin polypeptide increases at 37 degrees Celsius.

At higher temperatures there is an increase in the conductivity and permittivity of ionic salts. The proportional increase in conductivity is larger than the proportional increase in permittivity. Both the increase in conductivity and the increase in permittivity can contribute to an increase in the magnitude of thermoacoustic signals during thermoacoustic imaging yielding enhanced soft tissue contrast. In order to take advantage of this characteristic, prior to administering the contrast agent, the contrast agent can be preheated to a temperature at or above the subject's body temperature to improve the resolution of thermoacoustic imaging.

Alternately, a heating agent may be added to the mixture. It has been found that certain nanoparticles can absorb electromagnetic radiation and heat up. These nanoparticles may be made of gold, silver, platinum or iron-oxide or some combination thereof. The frequencies at which these nanoparticles heat up is dependent on the size of the nanoparticles. When these nanoparticles are included in the contrast agent mixture, during thermoacoustic imaging the nanoparticles heat up, which in turn heats up the contrast agent thereby to improve the resolution of thermoacoustic imaging. The heating agent may be in an amount of 10 grams per liter of thermoacoustic imaging contrast agent on a dilution thereof.

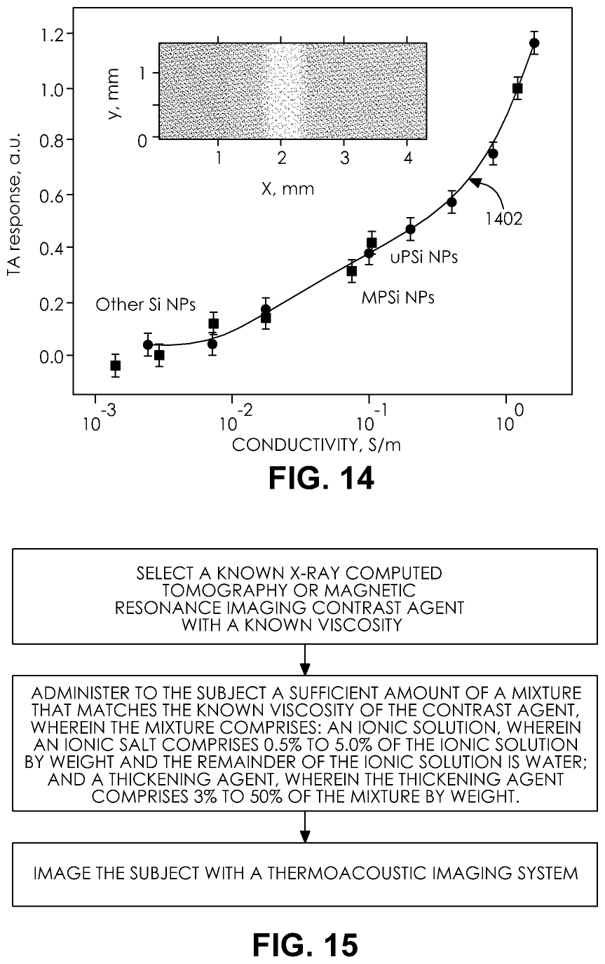

FIG. 14 shows thermoacoustic response as a function of saline solution conductivity with nanoparticle sized heating agents. Interpolation curve 1402 shows that the thermoacoustic response increases as the conductivity increases. The relationship between conductivity and the percentage of nanoparticles in solution is roughly linear in the range utilized for the purposes of the subject disclosure.

The images acquired during thermoacoustic imaging may be used to analyze soft tissue and/or vasculature, estimate blood flow and perfusion, and produce increased-contrast angiographic images. Non-limiting examples of tissues that may be analyzed (irradiated) in the provided methods include heart, kidney, lung, esophagus, thymus, breast, prostate, brain, muscle, connective tissue, nervous tissue, epithelial tissue, bladder, gallbladder, intestine, liver, pancreas, spleen, stomach, testes, ovaries, and uterus.

Perfusion of blood in and through tissue is related to the health of that tissue. Perfusion, being a general term, is more specifically characterized by parameters that include BF (blood flow), BV (blood volume), MTT (mean transit time), and PA (permeability-surface area product). BF is the volume flow of blood through the vasculature, comprising the large vessels, arteries, arterioles, capillaries, venules, veins, and venous sinuses. BF is usually normalized to a convenient volume of tissue and usually carries the unit of mL/min/100 g. BV is the fraction of a tissue of interest occupied by the blood in the vasculature (comprising the large vessels, arteries, arterioles, capillaries, venules, veins, and venous sinuses). It typically is expressed in units of mL/g or as a percentage. MTT recognizes that blood flows through multiple paths in tissue, so there does not exist a unique transit time from inlet to outlet, but rather a distribution of transit times. This distribution is represented by an average or mean transit time, being the mean of the distribution of transit times. The Central Volume Principle relates the parameters according to the relationship BF=BV/MTT. Variants and derived quantities (for example, dispersion of mean transit time) from these parameters also characterize perfusion tissue. As is known in the art, these measured parameters characterize tissue and can be used as a diagnostic to differentiate tissue types, e.g., healthy from diseased tissue, or necrotic from viable tissue.

In order to estimate blood flow parameters, specifically BF, BV, MTT, and PA, a bolus of the thermoacoustic imaging contrast agent is injected into the vasculature, either on the venous or arterial sides. The duration of the injection causes a time-varying concentration of the contrast agent, Ca(t), upstream of the region of interest within the subject to be imaged. The duration of the injection is typically short in comparison with the duration of the physiological events being measured, such as MTT. The curve describing Ca(t) becomes convolved with the dispersion of the contrast agent in its progression through the tissue and vasculature in the region of interest. The sequence of thermoacoustic images acquired during imaging using the thermoacoustic imaging system allows the concentration, Q(t), of the contrast agent in the tissue and vasculature over time to be measured. The arterial concentration of the contrast agent over time Ca(t) is also measured, and the blood flow parameters in the tissue of interest are computed by deconvolution of Q(t) and Ca(t) and analysis of the resulting concentration curve, as is known in the art. The measured and computed parameters can be presented as numerical results, can be displayed as parameter-vs.-time plots, can be images showing the spatial distribution of the parameters, or can be shown as images evolving in time (commonly called cineloops in the art).

The blood flow parameters (e.g., BF, BV, MTT and PA) may also be compared to the blood flow parameters (e.g., BF, BV, MTT and PA) measured in a healthy subject or a control tissue in the same subject. As will be appreciated by those of skill in the art, the thermoacoustic imaging contrast agent may be administered to the subject prior to the start of thermoacoustic imaging, alter the start of thermoacoustic imaging, or concurrently with thermoacoustic imaging. When the thermoacoustic imaging contrast agent is administered to the subject prior to the start of thermoacoustic imaging, a predefined period of time may be allowed to elapse before thermoacoustic imaging is commenced. The predefined period of time may for example be the estimated travel time of the thermoacoustic imaging contrast agent from its injection site in the subject to a region of interest within the subject to be imaged.

The images acquired during thermoacoustic imaging may also be used to classify the tissue according to its blood flow parameters and may use differences among the sequence of images to produce an angiogram image showing the blood vessels.

Furthermore, the images acquired during thermoacoustic imaging methods may be used to diagnose disease in a subject. Non-limiting examples of diseases that may be diagnosed using the methods of the invention include: cardiovascular disease, kidney disease, stroke, and cancer. Non-limiting examples of cancer that may be detected by the provided methods include adrenocortical carcinoma, anal cancer, appendix cancer, astrocytoma, atypical teratoid/rhabdoid tumor, basal cell carcinoma, bile duct cancer, bladder cancer, bone cancer, brain stem glioma, brain tumor, breast cancer, bronchial tumor, Burkitt lymphoma, carcinoid tumor, cervical cancer, chordoma, chronic lymphocytic leukemia, chronic myeloproliferative disorder, colon cancer, colorectal cancer, craniopharyngioma, cutaneous T-cell lymphoma, endometrial cancer, ependymoblastoma, ependymoma, esophageal cancer, Ewing sarcoma, extracranial germ cell tumor, extragonadal germ cell tumor, extrahepatic bile duct cancer, eye cancer, gallbladder cancer, gastric cancer, gastrointestinal cancer, germ cell tumor, gestational trophoblastic tumor, glioma, hairy cell leukemia, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, acute lymphoblatic leukemia, chronic lymphocytic leukemia, lip and oral cavity cancer, liver cancer, lung cancer, non-Hodgkin lymphoma, macroglobulinemia, osteosarcoma, medulloblastoma, melanoma, merkel cell carcinoma, mesothelioma, mouth cancer, mycosis fungiodes, myelodysplastic syndrome, multiple myeloma, nasal cavity and paranasal sinus cancer, nasopharyngeal cancer, non-small cell lung cancer, oral cancer, oropharyngeal cancer, osteosarcoma, ovarian cancer, ovarian epithelial cancer, pancreatic cancer, papillomatosis, parathyroid cancer, penile cancer, pharyngeal cancer, pituitary tumor, prostate cancer, rectal cancer, renal cell cancer, retinoblastoma, rhabdomycosarcoma, salivary gland cancer, sarcoma, skin cancer, small intestine cancer, soft tissue sarcoma, testicular cancer, throat cancer, thomoma, thymic carcinoma, thyroid cancer, urethral cancer, uterine cancer, vaginal cancer, and Wilms tumor.

Although the thermoacoustic imaging contrast agent described above comprises an ionic solution and a thickening agent, alternative thermoacoustic imaging contrast agents are available. For example, other solutions may be used with the thickening agent. Non-limiting examples of such solutions include: 1) Physiologic saline solution, which may include sodium chloride solution, other salt solution, or which may be a composite of several salts or other materials, such as may be commonly available and accepted for use for other medical application, such as Ringer's or Hartmann's solutions; 2) Hyperionic solutions, which exhibit more EM energy absorption to compared to blood. Hyperionic solutions may include: calcium chloride, calcium sulfate, calcium iodate, magnesium chloride, magnesium sulfate, copper sulfate, cuprous iodide, magnesium chloride, magnesium sulfate, magnesium phosphate, manganese sulfate, manganese chloride, potassium chloride, potassium iodate, potassium iodide, potassium sulphate, and sodium phosphate; 3) Hypoionic solutions or non-ionic solutions, which exhibit decreased EM energy absorption to radiation compared to blood and serves as a negative contrast. De-ionized water. Solutions containing the following salts in ionic concentrations less than physiological: calcium chloride, calcium sulfate, calcium iodate, magnesium chloride, magnesium sulfate, copper sulfate, cuprous iodide, magnesium chloride; 4) Low-conductivity isotonic solutions that do not promote cell shrinkage (plasmolysis) or rupture (cytolysis) due to osmotic difference from blood but exhibit lower EM energy absorption than blood and may be used as negative contrast agents (e.g., solutions containing molecules that do not dissociate in water, such as solutions of 5% mannitol, 5% dextrose, 2.5% glycerol, or similar solutions); and 5) Isotonic solutions, colloids, emulsions, suspensions, or mixtures that are modify the EM energy absorption of blood, and do not promote cell shrinkage (plasmolysis) or rupture (cytolysis) due to osmotic differences.

Alternatively, the contrast agents may comprise other materials in conjunction with the thickening agent such as: 1) blood plasma substitutes such as: Voluven, Haemaccel, Gelofusin. Suspensions or colloids of ferromagnetic and ferrimagnetic particles. Uncoated magnetite, Magnetic iron oxide particles bound in: starch, dextran, lipid, polyacrylic, elemental iron; 2) Suspensions or colloids of non-magnetic particles with a dielectric loss different from blood. Enzyme modified fats, lipids, oils, maltodextran, malt extract, corn sugar, corn syrup, safflower oil, glycerol; 3) Blood substitutes that exhibit a thermoacoustic response to EM energy different from blood. Perfluorocarbons, Hemopure, Oxygent, PolyHeme and Perftoran; and 4) Dyes that absorb EM energies in the red or infrared region of the spectrum, such as Indocyanine Green and Evan's Blue; and 5) Agents that exhibit a thermoacoustic response different from blood by having a different thermal expansion coefficient, speed of sound, heat capacity, or, in general, a different Gruneisen coefficient.

In all cases, the thermoacoustic imaging contrast agent is different from blood in the sense that it has a different thermoacoustic response to the applied EM energy, so it can be distinguished from blood or tissue by the difference in thermoacoustic signal produced. In some cases, the contrast agent moves out of the vasculature and into the interstitial space, thereby changing the endogenous absorption of the incident EM radiation. Depending on the makeup of the thermoacoustic imaging contrast agent, the physical mechanism that affords a difference in thermoacoustic response can be one or a combination of: a difference (an increase or decrease) in charge carrier density, such as ion density; a difference in dielectric absorption (loss tangent) (an increase or decrease); a difference in the speed of sound (increase or decrease); a difference in thermal expansion coefficient (an increase or decrease); a difference in heat capacity (an increase or decrease); or a difference in the molecular absorption (e.g., an optical or infrared dye).

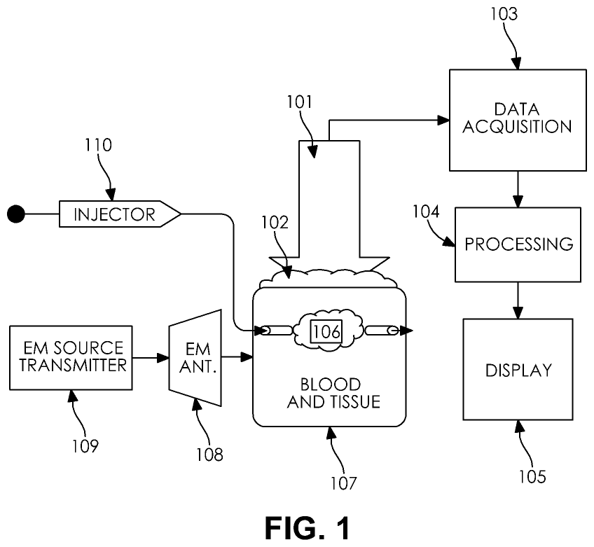

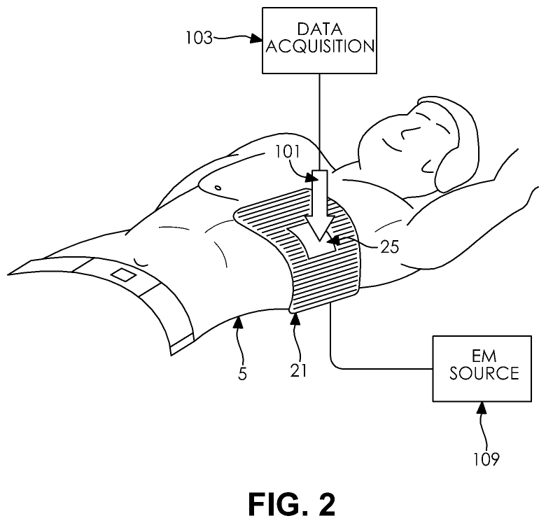

Turning now to FIGS. 1 and 2, an exemplary thermoacoustic imaging system to acquire thermoacoustic images of a subject having a thermoacoustic imaging contrast agent administered thereto is shown. In this embodiment, thermoacoustic imaging system comprises an ultrasound transducer 101 that is configured to receive thermoacoustic signals (acoustic pressure waves) from a region of interest 107 within a subject S that includes the tissue and/or vasculature 106 to be imaged. An acoustic coupling liquid or gel 102 is provided between the ultrasound transducer 101 and the skin or tissue surface of the subject S. An acoustic data acquisition unit 103 is coupled to the ultrasound transducer 101. A signal processor 104 is coupled to the data acquisition unit 103 and a display apparatus 105 is coupled to the signal processor 104. An EM applicator, transducer, transducer array, antenna, or antenna array (hereinafter “EM applicator”) 108 in close proximity to the subject S is coupled to an EM transmitter or power source (hereinafter “EM source”) 109. The EM source 109 and EM applicator 108 are configured to direct short pules of electromagnetic energy into the region of interest 107 of the subject S that includes the tissue and/or vasculature 106 to be imaged. An injector 110, which may be motorized and automatic or may be manually driven by an operator, is provided and is configured to inject a thermoacoustic imaging contrast agent such as those described above into the subject S.

The EM source 109 supplies energy at the appropriate power, frequency, and pulse shape to the EM applicator 108. During operation of the thermoacoustic imaging system, the EM source 109 and EM applicator 108 transmit pulses of electromagnetic energy into the region of interest 107, stimulating thermoacoustic signals that are detected by the ultrasound transducer 101. The thermoacoustic signals detected by the ultrasound transducer 101 are in turn digitized by the data acquisition unit 103. The digitized signals are then processed by the processor 104, and the results prepared and displayed 105.

The EM source 109 and EM applicator 108 are chosen 1) to provide a penetration depth in tissue suitable for a specific application, 2) to permit generation of individual pulses with a rise time short enough to produce acoustic pulses with detectable energy above one megahertz, and 3) to allow absorption to provide contrast. At least three specific regions of the EM spectrum are useful for this purpose: 1) near infrared light between 600 nm and 1000 nm, which has a useful penetration depth up to 2 cm; 2) microwave energy between 1 and 10 GHz, which exhibits good tissue contrast and penetration depth up to several centimeters; and 3) very high frequency and ultrahigh frequency radio waves between 26 MHz and 1000 MHz, which have frequencies high enough to produce the required short pulse rise time, and penetration depth of greater than several cm.

In one embodiment, the EM applicator 108 is in the form of an array of antennas. The array is driven in phase and amplitude to minimize to a practical extent the electromagnetic field present at the location of the ultrasound transducer, in order to reduce the excitation of the detector element(s) of the ultrasound transducer and consequent generation of spurious output to the data acquisition unit. The minimization of the EM field at the ultrasound transducer 101 is also assisted by reducing the induced signal entering the receiver electronics during EM pulse transmission, thus reducing or preventing risk of receiver damage or saturation and loss of sensitivity. An example is an array of a fixed geometry of discrete loop antennas; other examples include dipole, patch, microwave stripline, and transmission line antennas.

The EM applicator 108 may be embodied in a conformal, optionally flexible pad 21 that can be applied to the skin of the subject S as shown in FIG. 2. The pad may be provided with an aperture or acoustic window 25, through which the ultrasound transducer 101 can receive the thermoacoustic signals.

The pad 21 may be pre-formed for a specific body part or size of body pert, or it may be flexible to conform to a range of body surface shapes. During thermoacoustic imaging the ultrasound transducer 101 is placed at the acoustic window 25 provided in the pad 21, which may be located over the region of interest 107 in the subject S. The acoustic window 25 may be simply an opening in the pad 21 or 1 may be an acoustically-transparent membrane. In one embodiment, the pad 21 is advantageously designed to minimize the power density or the field strength of its emissions in the location of the window 26, to reduce interference with the ultrasound transducer 101.

In one embodiment using radiofrequency or microwave energy where magnetic contrast agents are used, the EM source 109 is configured to maximize the magnetic field within the region of tissue to be scanned.

In another embodiment using radiofrequency energy, where a contrast agent with high absorption due to ionic conductivity is used, the EM source 109 is configured to maximize the electric field within the region of tissue to be scanned.

In another embodiment using microwave energy, where a contrast agent with high dielectric absorption is used, the EM source 109 is configured to maximize the electric field within the volume of tissue to be scanned.

In another embodiment using radiofrequency or microwave energy, where a magnetic contrast agent is used (e.g., a contrast agent containing a ferromagnetic or ferrimagnetic molecule), the EM source 109 is configured to produce a circularly polarized electromagnetic field within the region of tissue to be scanned, with the objective to increase the difference in absorption between the contrast agent and the tissue.

In a further embodiment using microwave energy, where a ferromagnetic contrast agent is used, a complementary static magnetic field is used and the microwave frequency and magnetic field strength are adjusted to yield high absorption by the contrast agent, by exploiting the ferromagnetic resonance in the contrast agent.

In additional embodiments using radiofrequency or microwave energy, the EM source is advantageously in the form of a resonator with high quality factor to more efficiently couple the EM energy to the target absorbers.

Depending on environment, several configurations of the thermoacoustic imaging system are possible involving both fixed energy transmitting components or compact packaging enabling portability and point of care applications. In the fixed EM energy transmitting component configuration, the EM source 109 and EM applicator 108 are fixed, and the subject S is placed in proximity of the EM applicator. In a point of care application, the EM source and applicator are integrated into a compact deformable enclosure and may be placed in direct contact of the subject in proximity of the tissue to be imaged.

The following examples provided below are not meant to be limiting and are meant to demonstrate only certain embodiments:

### Example 1. In Vitro Experiments Demonstrating a Thermoacoustic Method

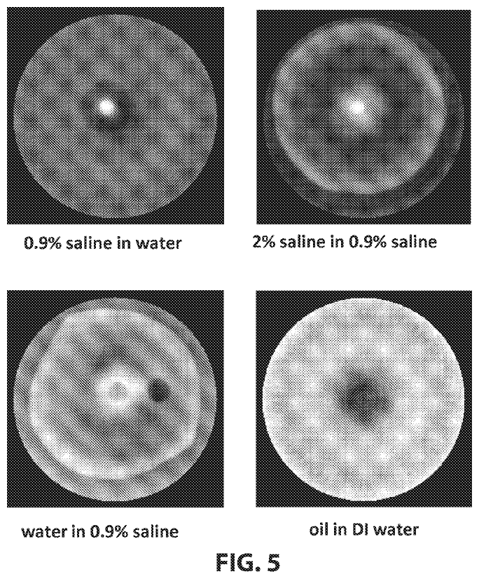

Experiments were performed in vitro to demonstrate the thermoacoustic imaging method using a variety of contrast agents. In each experiment, a suitable contrast agent was placed in a 2-mm tube that was surrounded by a second aqueous solution and irradiated using pulsed radiofrequency energy, and resulting thermoacoustic data were gathered (FIG. 5). The data show positive (i.e., increased thermoacoustic signal) and negative (i.e., decreased thermoacoustic signal) depending on the contrast agent and surrounding medium used in each experiment. The upper left panel of FIG. 5 shows increased signal due to an increase in ion concentration and thus, conductivity and energy absorption, in a 2-mm tube of physiological 0.9% saline versus the surrounding de-ionized water. The upper right panel of FIG. 5 shows increased signal resulting from irradiation of a 2-mm tube containing 2% saline within an environment of physiological 0.9% saline. The lower left panel of FIG. 5 shows decreased signal resulting from the irradiation of a 2-mm tube containing de-ionized water compared to the surrounding environment of physiological 0.9% saline. The lower right panel of FIG. 5 shows decreased signal from the irradiation of a 2-mm tube containing light mineral oil compared to the surrounding environment of de-ionized water, due to the relative lack of dielectric absorption in the predominantly non-polar oil compared to the polar molecules in water.

The sum of these data show the ability of the thermoacoustic imaging method to detect the presence of low toxicity contrast agents.

### Example 2. Spatial Resolution of Data Provided by a Thermoacoustic Method

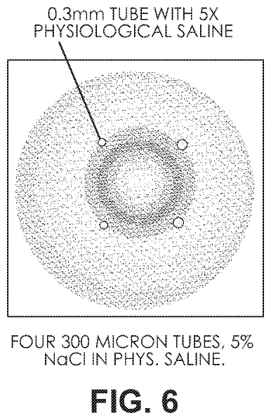

An in vitro experiment was performed to determine the spatial resolution of the data provided by the thermoacoustic methods. In this experiment, four 0.3-mm tubes containing 5× physiological saline (5% NaCl) were placed in an environment of physiological saline, the tubes were irradiated with pulsed radiofrequency energy, and the resulting thermoacoustic data were collected (FIG. 6). The resulting data demonstrate that the thermoacoustic method is able to detect sub-millimeter structures at depth with very high contrast.

Although embodiments have been described above with reference to the accompanying drawings, those of skill in the art will appreciate that variations and modifications may be made without departing from the scope thereof as defined by the appended claims.

What is claimed is:

1. A thermoacoustic imaging method comprising:
administering a thermoacoustic imaging contrast agent to a subject, the thermoacoustic imaging contrast agent having a viscosity higher than blood and substantially similar to a viscosity of a known computed tomography (CT) or magnetic resonance imaging (MRI) contrast agent; and
imaging the subject with a thermoacoustic imaging system.

2. The method of claim 1, wherein the thermoacoustic imaging contrast agent comprises an ionic solution and thickening agent mixture, wherein an ionic salt comprises 0.5% to 5.0% of the ionic solution by weight and the remainder of the ionic solution is water, and wherein the thickening agent comprises 3% to 50% of the mixture by weight.

3. The method of claim 1, wherein the mixture has a viscosity between 3 centipoise and 20 centipoise at 98 degrees Fahrenheit.

4. The method of claim 1, wherein the ionic salt is selected from the group consisting of sodium chloride, potassium chloride, magnesium chloride, calcium chloride, or some combination thereof.

5. The method of claim 1, wherein the thickening agent is a physiologically tolerable additive that increases the viscosity of the thermoacoustic imaging contrast agent.

6. The method of claim 5, wherein the thickening agent is selected from the group consisting of dextran, albumin, hydroxyethyl starch, or some combination thereof.

7. The method of claim 1, further comprising heating the thermoacoustic imaging contrast agent to a desired temperature prior to administering the thermoacoustic imaging contrast agent to the subject.

8. The method of claim 8, wherein the desired temperature is at or above the body temperature of the subject.

9. The method of claim 1, wherein the thermoacoustic imaging contrast agent further comprises a heating agent.

10. The method of claim 9, wherein the heating agent is in an amount of 10 grams per liter of thermoacoustic imaging contrast agent or dilution thereof.

11. The method of claim 10, wherein the heating agent is selected from the group consisting of gold nanoparticles, silver nanoparticles, platinum nanoparticles, iron-oxide nanoparticles, or some combination thereof.

12. The method of claim 1, further comprising, after administering the thermoacoustic imaging contrast agent to the subject, waiting a predefined period of time before imaging the subject.

13. The method of claim 12, wherein the predefined period of time is an estimated travel time of the thermoacoustic imaging contrast agent from is injection site in the subject to a region of interest within the subject to be imaged.

14. A thermoacoustic imaging method comprising:
administering to a subject a sufficient amount of a mixture that matches the viscosity of a known computed tomography or magnetic resonance imaging contrast agent, wherein the mixture comprises, an ionic solution, wherein an ionic salt comprises 0.5% to 5.0% of the ionic solution by weight and the remainder of the ionic solution is water, and a thickening agent, wherein the thickening agent comprises 3% to 50% of the mixture by weight; and
imaging the subject with a thermoacoustic imaging system.

15. The method of claim 14, wherein the mixture has a viscosity between 3 centipoise and 20 centipoise at 98 degrees Fahrenheit.

16. The method of claim 14, wherein the ionic salt is selected from the group consisting of sodium chloride, potassium chloride, magnesium chloride, calcium chloride, or some combination thereof.

17. The method of claim 14, wherein the thickening agent is a physiologically tolerable additive that increases the viscosity of the thermoacoustic imaging contrast agent selected from the group consisting of dextran, albumin, hydroxyethyl starch, or some combination thereof.

18. The method of claim 14, wherein the thermoacoustic imaging contrast agent further comprises a heating agent selected from the group consisting of gold nanoparticles, silver nanoparticles, platinum nanoparticles, iron-oxide nanoparticles, or some combination thereof.

19. A thermoacoustic imaging contrast agent comprising a mixture that matches the viscosity of a known computed tomography or magnetic resonance imaging contrast agent, wherein the mixture comprises, an ionic solution, wherein an ionic salt comprises 0.5% to 5.0% of the ionic solution by weight and the remainder of the ionic solution is water, and a thickening agent, wherein the thickening agent comprises 3% to 50% of the mixture by weight.

20. The thermoacoustic imaging contrast agent of claim 19, wherein the mixture has a viscosity between 3 centipoise and 20 centipoise at 98 degrees Fahrenheit, the ionic salt is selected from the group consisting of sodium chloride, potassium chloride, magnesium chloride, calcium chloride, or some combination thereof, and the thickening agent is selected from the group consisting of dextran, albumin, hydroxyethyl starch, or some combination thereof.
